(19) United States
(12) Patent Application Publication
(10) Pub. No.: US 2006/0172021 A1
(43) Pub. Date: Aug. 3, 2006

(54) Herbal compositions for the prevention or treatment of symptoms of stress and infection

(76) Inventor: Scott Alexander Moffett, Bangkok (TH)

Correspondence Address: FOLEY & LARDNER LLP, 111 HUNTINGTON AVENUE, 26TH FLOOR, BOSTON, MA, 02199-7610, US

(21) Appl. No.: 11/328,439

(22) Filed: Jan. 9, 2006

Related U.S. Application Data
(60) Provisional application No. 60/642,741, filed on Jan. 10, 2005.

(51) Int. Cl. A61K 36/906; A61K 36/67; A61K 36/53; A61K 36/185

(52) U.S. Cl. 424/725; 424/773; 424/745; 424/734; 424/756

(57) ABSTRACT

The present invention provides herbal compositions useful for the prevention or treatment of the symptoms of stress and/or infection in a subject in need thereof. The herb-containing compositions of the invention can be formulated in a dry delivery system, liquid delivery system, or a controlled-release vehicle. The herb-containing compositions of the invention are formulated as dosage unit forms which include a tablet; dry powder; capsule; and caplet. The present invention also provides methods for the prevention, alleviation or treatment of symptoms of stress or infection in a subject.

### RELATED APPLICATIONS

The present application is related to U.S. provisional application U.S. Ser. No. 60/642,741, filed on Jan. 10, 2005, to which it claims the benefit of priority and the contents of which are incorporated herein by reference in its entirety.

### FIELD OF THE INVENTION

The present invention relates to herbal compositions for the prevention, alleviation or treatment of symptoms of stress and/or infection in a subject in need thereof.

### BACKGROUND OF THE INVENTION

Every year, millions of people world-wide get sick with the flu (influenza) and cold. It is estimated that the average American adult suffers two to three colds a year; the average young child has as many as nine. The fever, exhaustion, and aches and pains of the flu can be debilitating for a week or two, but for the elderly and those with compromised immune systems the flu can be much more serious. An estimated 100,000 hospitalizations and about 20,000 deaths occur each year from the flu or its complications in the US alone.

The common cold is caused by any one of 200 different viruses. When infection occurs, the walls of the respiratory tract swell and produce excess mucus, giving rise to the typical cold symptoms. Symptoms range from sore throat, running nose, nasal congestion, watery eyes to hacking cough, headache, and fever. Most colds run their course in 7-10 days. Recurrent colds (almost constantly suffering) may indicate a lowered immune capacity and too much stress and often not enough sleep.

Stress can lower the body's resistance to infectious diseases (Yang and Glaser (2002) Int Immunopharmacol. 2:315-24; Cohen S et al., (1991) New Engl J Med. 325:606-12; Miller G E et al., (2004) Psychosom Med. 2004;66:215-23; Hunzeker J. et al., (2004) 18:526-35; Burns V E et al., (2003) J Psychosom Res. 2003;55:569-72). Stress is a natural response to everyday physical, emotional and environmental demands.

Many natural herbal remedies have been advanced for the prevention and treatment of the common cold and flu. For example, Echinacea, garlic, onion, ginger, goldseal and peppermint have all been used in natural herbal remedies for the prevention and treatment of viral and bacterial infection in humans as well as the alleviation of the symptoms related thereto. The potency and effectiveness of natural remedies depends, in part, on the quality and uniformity of prophylactic or therapeutic effect active herbal constituents. Crude herbal extracts and non-standardized herbal formulations can yield less predictable biological effect due to variability in the concentrations and/or stability of bioactive constituents. Furthermore, impurities in herb-containing preparations can yield unwanted side-effects in a subject. Therefore, there is a need for the identification of new herb-containing compositions preparations with standardized active constituents and formulation optimized for the potency of bioactive constituents which are useful for the prevention or treatment of symptoms of stress and/or infection in a subject in need thereof.

### BRIEF SUMMARY OF THE INVENTION

The present invention provides herbal compositions useful for the prevention, alleviation or treatment of the symptoms of stress and/or infection. The herb-containing compositions of the invention can be formulated in a dry delivery system, liquid delivery system, or a controlled-release vehicle. The herb-containing compositions of the invention are formulated as dosage unit forms which include a tablet; dry powder; capsule; and caplet. The present invention also provides methods for the prevention, alleviation or treatment of symptoms of stress or infection in a subject.

### DETAILED DESCRIPTION OF THE INVENTION

It is to be appreciated therefore that certain aspects, modes, embodiments, variations and features of the invention described below in various levels of detail in order to provide a substantial understanding of the present invention. In general, such disclosure provides beneficial herb-containing compositions, combinations of such compositions with other dietary supplement compositions, and related methods of producing and using same.

The various aspects of the present invention relate to therapeutic or prophylactic uses of certain particular herb-based compositions in order to prevent, alleviate, or treat one or more symptoms of stress and/or infection in a subject. Accordingly, various particular embodiments that illustrate these aspects follow.

Definitions

It is to be appreciated that the various modes of treatment or prevention of medical conditions as described are intended to mean “substantial”, which includes total but also less than total treatment or prevention, and wherein some biologically or medically relevant result is achieved.

A “subject,” as used herein, is preferably a mammal, such as a human, but can also be an animal, e.g., domestic animals (e.g., dogs, cats and the like), farm animals (e.g., cows, sheep, pigs, horses and the like) and laboratory animals (e.g., rats, mice, guinea pigs and the like).

An “adaptogen” as used herein refers to an herb that increases a subject's overall resistance to stress when it is administered to the subject.

The term “standardized” as used herein refers to a process that yields a consistent amount of an active plant component in each oral dosage unit of the herb-containing compositions of the invention.

The term “extract” refers to an herb in the form of a concentrated powder or liquid. Generally extracts are derived by the extraction of select herb parts using only pure water & alcohol but not toxic solvents.

An “effective amount” of a compound, as used herein, is a quantity sufficient to achieve a desired therapeutic and/or prophylactic effect, for example, an amount which results in the prevention of or a decrease in the symptoms associated with a disease that is being treated. The amount of compound administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. Typically an effective amount of the compositions of the present invention are an amount sufficient for achieving a therapeutic or prophylactic effect.

It is advantageous to formulate oral compositions in dosage unit form for ease of administration and uniformity of dosage. Dosage unit form (a.k.a., dosage unit) as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the dietary supplement and the particular therapeutic effect to be achieved, and the limitations inherent in the art of compounding such an active compound for the treatment of individuals. The pharmaceutical compositions can be included in a container, pack, or dispenser together with instructions for administration. In some embodiments of the invention, the herb-containing compositions of the invention are formulated as dosage unit forms units which include a tablet; dry powder; capsule; and caplet. Typically, an oral dose is taken two-times to four-times daily, until symptom relief is apparent. Administration of the composition of the invention in some embodiments is greater than at least about 30 days. In one embodiment, a 500 mg oral dose is administered to a subject two-times to three-time daily after meals. The compounds of the present invention can also be administered in combination with each other, or with one or more additional therapeutic compounds.

The content of the references cited in this application are incorporated by reference in their entireties.

Herb-Containing Compositions of the Invention

One object of the present invention is to provide new and useful compositions that prevent, alleviate or treat one or more symptoms of stress in a subject, e.g., physical, emotional, and mental stress. That is, the present invention provides herb-containing compositions that function as adaptogens when administered to a subject. In one embodiment, the herb-containing compositions of the invention are used to prevent, alleviate or treat one or more symptoms of stress in a subject. Symptoms of physical stress in a subject include, but are not limited to, e.g., heart pounding, headaches, sweaty palms, indigestion, skin breaks out, shortness of breath, holding breath, cold hands, sleeplessness, sleep too much, fatigue, nausea, diarrhea, tight stomach, tight muscles, and pain, loss of libido. Symptoms of emotional stress in a subject include, but are not limited to, e.g., moody, Irritability, depressed anxious, lack of sense of humor, abrasive, hostile, nervous, and emotional. Symptoms of mental stress in a subject include, but are not limited to, e.g., forgetfulness, loss of concentration, poor judgment, disorganized, fuzzy perception, confused, lack of interest, math errors, stop thinking, diminished fantasy life and negative self-talk.

Another object of the present invention is to provide compositions that assist the body's natural defense system(s) to alleviate the symptoms associated with infection, e.g., viral or bacterial. In one embodiment, the herb-containing composition of the present of the present invention are used to prevent, alleviate or treat one or more symptoms of infection in a subject, e.g., viral and bacterial infection. In one embodiment, the herb-containing compounds of the invention are used to prevent, alleviate or treat one or more symptoms of a common cold in a subject. The common cold is a viral infection that can be caused by several types of viruses. Symptoms of the common cold include, but are not limited to, e.g., runny nose, congestion, sneezing, weakened senses of taste and smell, scratchy throat, cough, and fever.

In one embodiment, the herb-containing compositions of the invention are used to prevent, alleviate or treat one or more symptoms of influenza in a subject. Influenza (a.k.a, the flu) is an infection of the respiratory system caused by the influenza virus. There are three types of influenza virus: A, B, and C. Types A and B are the most severe. The viruses change constantly and different strains circulate around the world every year. Type C causes either a very mild illness, or has no symptoms at all. It does not cause epidemics and does not have the severe public health impact that influenza types A and B do. Symptoms of the flu include, but are not limited to, e.g., temperature of 101° F. or above, cough, muscle ache, headache, sore throat, chills, tiredness, feeling lousy all over, and vomiting.

In one embodiment, the herb-containing compositions of the invention are used to prevent, alleviate or treat one or more symptoms of bacterial infection in a subject. Symptoms of bacterial infection include, but are not limited to, e.g., fever that may return repeatedly for months to years; abdominal pain; vomiting; diarrhea; bone and joint pain, swollen lymph nodes that fill with pus, headache, confusion, lethargy, discharge, cough; general weakness; chills; chest pain; shortness of breath; tissue abscesses; skin sores, chills; vomiting; headache; back and joint pain; skin and joint swelling

Yet another object of the present invention is to formulate a composition in the form of a tonic or capsule, with the beneficial effects of alleviating the symptoms of stress and/or infection that is of a sufficient concentration that may be easily implemented as part of a daily supplement regime. The invention is also directed to a method of alleviating the symptoms of stress and/or infection in a subject by administering the herb-containing compositions to a subject in need thereof. In one embodiment of the method of the invention the herb-containing composition is administered as a dosage unit form. In one embodiment of the method of the invention, the dosage unit form is a dietary supplement. These and other objects of the present invention will become more readily appreciated and understood from a consideration of the following detailed description of the present invention.

The present invention provides herb-containing compositions formulated with standardized components that reduce the symptoms of physiological stress and/or infection with little, if any, counteracting side-effects. That is, in some embodiments, the herb-containing composition of the invention is standardized for select biologically active agents, e.g., andrographolides, ursolic acid, and withanolides. The use of a standardized preparations of herb components is advantageous because the inter-batch variation of biologically active constituents is reduced, thus the herb-containing composition of the present invention yields more consistent preventative or therapeutic effect. Also, plant extract which are typically the ingredients of natural dietary supplements, generally have their respective undesirable side-effects that also need to be properly balanced in an overall formulation that accomplishes a dilution of these individual undesirable effects. The foregoing problems of batch-to-batch variation and undesirable side effects, among others, have been resolved by the present invention. Specifically, the present invention provides compositions containing appropriate mixtures of plant extracts that safely and reproducibly alleviate the symptoms of physiological stress infection while at the same time diluting the undesirable side-effects associated with the individual extracts that make up the formulation.

The mixture of the present invention is comprised of at least the following five (5) ingredients, which are listed in the Table 1 along with the desired amounts of each ingredient. An asterisk (*) indicates that the herbal component is a standardized herbal component.

TABLE 1
Standardized Herbal Composition of the Present Invention

| | Range A | Range B | Range C |
|---|---|---|---|
| | (mg/dosage | (mg/dosage | (mg/dosage |
| Component | unit) | unit) | unit) |
| Andrographis extract | 50-250 mg* | 100-200 mg* | 150 mg* |
| (Andrographis | (10-50 mg | (20-40 mg | (30 mg |
| paniculata) | equivalent | equivalent | equivalent |
| 20% (wt/wt) | andro- | andro- | andro- |
| andrographolides | grapholides) | grapholides) | grapholides) |
| Holy basil extract | 125-225 mg* | 150-200 mg* | 175 mg* |
| (Ocimum sanctum) | (2.5-4.5 mg | (3.0-4.0 mg | (3.5 mg* |
| 2% (wt/wt) | equivalent | equivalent | ursolic |
| ursolic acid | ursolic | ursolic | acid) |
| | acid) | acid) | |
| Ashwagandha root | 50-150 mg* | 75-125 mg* | 100 mg* |
| extract | (2.5-7.5 mg | (3.75-6.25 mg | (5 mg |
| (Withania | equivalent | equivalent | equivalent |
| somnifera) | withanolides) | withanolides) | withanolides) |
| 5% (wt/wt) | | | |
| withanolides | | | |
| Trikatu extract | 10-50 mg | 15-40 mg | 25 mg |
| (Equal parts | | | |
| of Long | | | |
| pepper fruit | | | |
| (Piper longum), | | | |
| Black pepper fruit | | | |
| (Piper nigrum), | | | |
| and Ginger root | | | |
| (Zingiber | | | |
| officinale) | | | |
| extracts | | | |
| Holy basil | 10-100 mg | 30-70 mg | 50 mg |
| leaf powder | | | |

As shown in Table 2, many of the plant extracts listed have the possibility of undesirable side-effects at high doses. However, by using a combination of these five ingredients in the respective amounts as listed in Table 1 to formulate an herb-containing composition (e.g., an oral dietary supplement), the desirable effects are expected to be additive and in certain embodiments, even synergistic. The overall effect expected, then, is to prevent or alleviate symptoms of stress and/or infection in a subject. Furthermore, the likelihood of observing the undesired side-effects listed for each respective ingredient is more remote as each extract is diluted out by a factor of up to five (5).

TABLE 2
Potential Side-Effects of Select Herbal Components

| Component | Possible undesired Effects |
|---|---|
| Andrographis | Mice that received oral extracts of |
| extract | Andrographis (10 g/kg body weight) |
| | once a day for seven days, were lethargic |
| | and less active. The use of AP has been |
| | associated with allergic reactions ranging |
| | from minor skin rashes to more serious |
| | anaphylaxis, which is a potential problem |
| | at high doses. Whether or not these reactions |
| | are due to AP per se or other matter in |
| | herbal preparations is not clearly understood. |
| | Andrographis has clear antifertility |
| | as well as pregnancy-terminating effects. |
| | Andrographis given as dry leaf powder |
| | (105 mg. of powder/kg body weight) each day for |
| | 60 days, stopped spermatogenesis (development |
| | and maturation of sperm cells). Studies by Zoha |
| | and colleagues, also in India, reported |
| | antifertility effects on female mice. Studies |
| | done in cultured human placental tissue |
| | showed that andrographolide sodium succinate |
| | (derived from AP) was effective in inhibiting |
| | human progesterone production. This hormone |
| | is necessary for pregnancy to be successful. |
| | The form of AP used was tissue specific, |
| | meaning it only affected the tissue it was |
| | intended for. There were no detrimental effects |
| | on other normal human tissue, even at the |
| | highest doses tested. The researchers concluded |
| | that the derivatives appeared to be promising |
| | contraceptives. Other studies in female mice |
| | using dehydroandrographolide indicated that |
| | the dose required to affect pregnancy was |
| | 250 mg/kg. of body weight. This amount of pure |
| | compound would not be found in the 105 mg/kg. |
| | body weight dose of AP given to male animals or |
| | the 2 g (2,000 mg/kg body weight) given to the |
| | female animals in the studies described above. |
| | Thus, it appears unlikely that the active |
| | compound in AP causing infertility is a member |
| | of the andrographolide series of compounds. |
| Holy basil | Holy basil can have antispematogenic effects |
| extract | (Seth 1981 Indian J Exp Biol. 1981 October; |
| | 19(10): 975-6; Kasinathan et al., Indian J Exp |
| | Biol. 1972 January; 10(1): 23-5; Batta et al., |
| | Indian J Med Res. 1971 May; 59(5): 777-81). |
| | Holy basil can cause allergic inflammatory |
| | response, e.g., contact dermatitis (Cutis. |
| | 1993 November; 52(5): 288-90.) |
| Ashwagandha | May increase estrogen leading to abortion. |
| root extract | |
| Trikatu extract | With large doses of some of the active |
| | principles of Zingiber officinale CNS |
| | depression has been noted in animals. |
| | Piper nigrum may cause digestive inflammation. |

Further, it is expected that the above-described ingredients, in the amounts listed in Table 1, will provide a combined remedy to prevent or alleviate symptoms of stress and/or infection in a subject that may be processed into a dosage unit for oral administration. This dosage unit, then, can be administered, as a tablet, capsule, gel cap, pellet (globule), or other carrier suitable for oral administration. Alternatively, the mixture can be made available as a powder to be mixed with a suitable liquid, such as water, to form a tonic. In one embodiment, the formulation of the herb-containing composition of the invention is an oral dietary supplement. As such, an efficient, proper, and effective balance of these ingredients can be formulated as to provide a composition that can be administered as a suitable dietary supplement.

In one embodiment, the herb-containing composition of the invention contains at least five (5) of the components (e.g., ingredients) summarized in Table 1. The herb-containing composition of the invention can contain the components summarized in Table 1 in any quantity, or combination, suitable to give the desired stress or infection preventative, therapeutic, or alleviating effect.

In one embodiment, the herb-containing composition of the invention contains an A. paniculata extract standardized for at least about 20% (wt/wt) andrographolides. In another embodiment, the herb-containing composition of the invention contains an A. paniculata extract standardized for at least about 30% (wt/wt) andrographolides. In another embodiment, the herb-containing composition of the invention contains A. paniculata extract standardized for at least about 40% (wt/wt) andrographolides. In another embodiment, the herb-containing composition of the invention contains A. paniculata extract standardized for at least about 50% (wt/wt) andrographolides.

In one embodiment, the herb-containing composition of the invention contains an O. sanctum extract standardized for at least about 2.0% (wt/wt) ursolic acid. In another embodiment, the herb-containing composition of the invention contains an O. sanctum extract standardized for at least about 3.0% (wt/wt) ursolic acid. In another embodiment, the herb-containing composition of the invention contains O. sanctum extract standardized for at least about 4.0% (wt/wt) ursolic acid. In another embodiment, the herb-containing composition of the invention contains O. sanctum extract standardized for at least about 5.0% (wt/wt) ursolic acid.

In one embodiment, the herb-containing composition of the invention contains an W. somnifera extract standardized for at least about 2.0% withanolides. In another embodiment, the herb-containing composition of the invention contains an W. somnifera extract standardized for at least about 3.0% withanolides. In another embodiment, the herb-containing composition of the invention contains W. somnifera extract standardized for at least about 4.0% withanolides. In another embodiment, the herb-containing composition of the invention contains W. somnifera extract standardized for at least about 5.0% withanolides.

In one embodiment, an oral dosage unit of the herb-containing composition of the invention contains A. paniculata extract, O. sanctum extract, W. somnifera extract, Trikatu extract and holy basil leaf powder in concentrations as described in Range A of Table 1. In another embodiment, an oral dosage unit of the herb-containing composition of the invention contains A. paniculata extract, O. sanctum extract, W. somnifera extract, Trikatu extract and holy basil leaf powder in concentrations as described in Range B of Table 1. In yet another embodiment, an oral dosage unit of the herb-containing composition of the invention contains A. paniculata extract, O. sanctum extract, W. somnifera extract, Trikatu extract and holy basil leaf powder in concentrations as described in Range C of Table 1.

In some embodiments, the herb-containing compositions of the invention contain rice flour, maltodextrin or silicon dioxide. In one embodiment, the herb-containing composition of the invention contains from about 0.001% (wt/wt) to about 20% (wt/wt) rice flour. In one embodiment, the herb-containing composition of the invention contains from about 0.001% (wt/wt) to about 5% (wt/wt) rice flour. In one embodiment, the herb-containing composition of the invention contains from about 0.001% (wt/wt) to about 1% (wt/wt) rice flour. Silicon dioxide is a natural mineral used to keep Pegasus dry. In one embodiment, the herb-containing composition of the invention contains from about 0.001% (wt/wt) to about 20% (wt/wt) silicon dioxide. In one embodiment, the herb-containing composition of the invention contains from about 0.001% (wt/wt) to about 5% (wt/wt) silicon dioxide. In one embodiment, the herb-containing composition of the invention contains from about 0.001 % (wt/wt) to about 1 % (wt/wt) silicon dioxide. Maltodextrin is used in small amounts to adjust the percentage of withanolides. In one embodiment, the herb-containing composition of the invention contains from about 0.001% (wt/wt) to about 20% (wt/wt) maltodextrin. In one embodiment, the herb-containing composition of the invention contains from about 0.001 % (wt/wt) to about 5% (wt/wt) maltodextrin. In one embodiment, the herb-containing composition of the invention contains from about 0.001 % (wt/wt) to about 1 % (wt/wt) maltodextrin.

Biological/Pharmacological Properties and Uses of Components of the Compositions of the Invention

Andrographis (A. paniculata) leaf is traditionally used in Ayruveda in the treatment of flu (Nadkarni A K. (1976) Indian Materia Medica. Vol.1. Bombay, India: Popular Prakashan, 01-3 rni, 1976), fever and general debility (Vedavathy S, Rao K N. (1991), J Ethnopharmacol., 33:193-6.) and in traditional Chinese medicine in treating flu with fever, cough, and sore throat. (Tu G et al. (eds.)(1992) Pharmacopoeia of the People's Republic of China. Guangzhou, China: Guangdong Science and Technology Press, p. 90). Andrographolides are a group of active plant compounds in Andrographis thought responsible for many of its prophylactic and therapeutic benefits. Andrographis has shown immunostimulating, (Puri A et al. (1993) J Nat Prod., 56:995-9) fever-reducing (comparable to aspirin), (Vedavathy and Rao, (1991) J Ethnopharmacol., 33(1-2):193-6) and other beneficial effects. Double-blind, placebo-controlled clinical trials of Andrographis in the treatment of uncomplicated upper respiratory tract infections (colds, flu, sore throat) have shown significant results. (Cáceres D D et al. (1999) Phytomedicine. 1999;6:217-23; Melchior J et al. (1996/97) Phytomedicine., 3:315-8). Andrographis has been clinically tested in over 1300 subjects with cold or flu and found effective, whether taken alone or combined with other herbal ingredients. (Coon J T, Ernst E. (2004) Planta Med., 70:293-8; Poolsup N et al. (2004) J Clin Pharm Ther., 29:37-45). Symptoms have improved in as little as two days, including nasal secretions, soreness of the throat, fatigue, sleep disturbance, and the frequency and intensity of coughing. (Cáceres D D et al. (1999) Phytomedicine, 6:217-23). Andrographis produced significantly fewer sick leave days, less severity of symptoms, and faster recovery from the common cold compared to placebo (Melchior J et al. (1996/97) Phytomedicine, 3:315-8). The herb-containing composition of the present invention is therefore useful to prevent, treat or alleviate the symptoms of stress and/or infection in a subject.

Holy basil (O. sanctum) is related to common basil and is traditionally regarded in India as a sacred plant believe to protect the household (Chopra D, Simon D. (2000) The Chopra Center Herbal Handbook. New York, N.Y.: Three Rivers Press, pp. 158-61). In the Ayurvedic tradition, the leaves are used to treat colds (Jain S K and DeFilipps R A. (1991) Medicinal Plants of India. Vol.1. Algonac, MI: Reference Publications; 1991:372-3), cough, and fever (Kirtikar K R, Basu B D, An ICS. (1994) Indian Medicinal Plants. Vol. 3. Dehra Dun, India: Bishen Singh Mahendra Pal Singh, pp. 1965-8), and to prevent flu (Nadkarni A K. (1976) Indian Materia Medica. Vol. 1. Bombay, India: Popular Prakashan, pp. 865-7). Research has shown that Holy basil holds numerous potential health benefits, including immunostimulant (Godhwani A, et al. (1988) J Ethnopharmacol., 24:193-8), fever-reducing (Godhwani A, et al. (1987) J Ethnopharmacol., 21:153-63), antimicrobial (Ahmad I and Beg A Z. (2001) J Ethnopharmacol., 74:113-23), antistress (Sembulingam K, et al. (1997) Indian J Physiol Pharmacol., 41:139-43; Sembulingam K, et al. (1999) Indian J Physiol Pharmacol., 43:137-40 (letter)), physical endurance-increasing (more than ginseng) (Singh N, et al. (1991) Indian J Pharmacol., 23:99-103), and antidepressant-like activity (Sakina M R, et al. (1990) J Ethnopharmacol., 28:143-50). In clinical studies on Holy basil, improvements have been reported in the treatment of acute viral encephalitis (Das S K, et al. (1983) Antiseptic., 80:323-6) and tropical pulmonary eosinophilia (Sharma R, et al. (1987) J Res Ed Indian Med. 1987;6:11-7). Ursolic acid is an active plant compound found in Holy basil thought responsible for some of the prophylactic and therapeutic benefits of this herb. The herb-containing composition of the present invention is therefore useful to prevent, treat or alleviate the symptoms of stress and/or infection in a subject.

Ashwagandha (W. somnifera; Indian ginseng) is renowned as an herbal adaptogen. In the Indian Ayurvedic tradition, the root is used as a Rasayana, a dietary supplement or practice promoting rejuvenation, mental and physical health, as well as providing a defense against aging and challenging environmental factors (Bhattacharya A et al. (2001), J Ethnopharmacol., 74:1-6). Research has shown that Ashwagandha root extracts stimulate the immune system (Dhuley J N. (1997) J Ethnopharmacol., 58:15-20; Dhuley J N. (1998) 20:191-8;

Ziauddin M, et al. (1996) 50:69-76), protect against stress-induced suppression of immune cell function, and show antidepressant-like activity and other antistress effects (Bhattacharya S K and Muruganandam A V (2003) Pharmacol Biochem Behav., 75:547-55). The root has also been shown to protect brain cells from oxidative damage caused by exposure to stressors (Jain S, et al. (2001) Phytotherapy Research, 15:544-8). Ashwagandha reduced the number of neurons damaged due to oxidative stress by 80%—an effect attributed to protection against stress-induced free radicals in key areas of the brain (Shukla S D, et al. (2000). Indian Journal of Experimental Biology, 38:1007-13). In a placebo-controlled study, men and women given an extract of Ashwagandha root showed improved mental skills by performing better in reaction times, mental arithmetic and logical deductions (Karnick C R. (1991) Indian Medicine, 3(2-3):1-5). Withanolides are a group of active plant compounds in Ashwagandha thought responsible for some of its prophylactic and therapeutic benefits of this herb. The herb-containing composition of the present invention is therefore useful to prevent, treat or alleviate the symptoms of stress and/or infection in a subject.

Trikatu or the ‘three pungent spices’, is a traditional formula used to treat colds (Dash V B, Junius A M M (1983). A Handbook of Ayurveda. New Delhi, India: Concept Publishing, pp. 195-196) and to enhance digestion and appetite (Simon D (1997). The Wisdom of Healing. New York, N.Y.: Harmony Books, p. 149). It consists of equal parts of Long pepper fruit (Piper longum), Black pepper fruit (Piper nigrum), and Ginger root (Zingiber officinale). Laboratory studies indicate that Trikatu slows the passage of substances through the intestine which would allow more thorough digestion (Annamalai A R and Manavalan R (1990). Indian Drugs., 27:595-604). Through its slowing effects on liver enzymes involved in drug metabolism (Atal C K et al. (1995) J Pharmacol Exp Ther., 232:258-62), it can also enhance the bioavailability of other herbal products taken at the same time (Shoda G et al. (1998) Planta Med., 64:353-6; Lambert J D et al. (2004) J Nutr., 134:1948-52). The herb-containing composition of the present invention is therefore useful to prevent, treat or alleviate the symptoms of stress and/or infection in a subject.

Ginger root (Zingiber officinale) is derived from a herbaceous rhizomatous perennial, up to 90 cm in height when fully grown (Raghunathan, K. and R. Mittra: Pnarmacognosy of indigenous Drugs. Central Council for Research in Ayurveda & Siddha. New Delhi. (1982)).

The herb develops several lateral shoots in clumps. The rhizomes are aromatic, thick lobed pale yellow, bearing simple alternate (listichotis narrow, oblong lanceolate leaves. Ginger contains 1-2% volatile oil and 5-8% resinous matter, starch and mucilage. The oil of ginger is a mixture of over 24 constituents, consisting of monoterpenes (phellandrene,(+)-carnphene, cineolc, citral and borneol) and sesquiterpenes etc (zingiberine, and hisabolene) (Trease, G. E. and W. C. Evans: Pharmacognosy, ELBS/Ballicre Tindall, Eastbourne (1983)). The pungent component is gingerol (Smith, R. M. and J. M. Robinson, Phytochemistry 20: 203 (1981); Slucki, K. and E. Steinegger: Planta Med. 39: 274 (1980))—‘formed in the plant from phenylalanine, nialoiiate and hexanoate. (Deniff, J. et al., J. Chem. Soc. 1. 1267 (1980)). Minor constituents of an extract are gingreniols, methylgingediol, gingeryldiacetates and methyl gingediacetates (Harvey, D. J., J. Chromatogr. 212: 75). Anti-inflammatory activity in carrageenin-induced rat paw oedema has been shown with ginger root. The active principles—(Harvey, D. J., J. Chromatogr. 212: 75)—gingerol, (Harvey, D. J., J. Chromatogr. 212: 75)—and (Babu, S. R. Rheumatism IS: 24 (1982))—dehydrogingerdione and (Harvey, D. J., J. Chromatogr. 212: 75) and (Babu, S. R., Rheumatism IS: 24 (1982))—gingerdione were shown to be potent inhibitors of prostaglandin synthesis’—confirming the mechanism of anti-inflammatory effect. The antirheumatic effects were further confirmed by other investigators (Kishore, P. et al., Rheumatism 19. 476 (1982); Babu, S. R., Rheumatism IS: 24 (1982)). Antihistaminic activity has also been shown in vitroll. The plant inhibits the virion toxic factor production in infected chorioallantoin membrane and also inhibits the growth of W.M.—25d malignant cell-line (Battar, 0. P. Ind. J. Exp. Biol. 20: 572 (1982)). Cardiac inotropic activity has been shown in dogs (Koho, J. P.: Kokai Tokyo 82: 59: SW (1982) Chem. Abstr. 97: 33378 k (1982)) and guinea pigs (Ohijumi, Y. et al.: J. Pharm. Sci. 71: 1174 (1982)). Ginger was shown to have significant antiemetic and antivertigo effects like Dramamine. It has been listed effectively along with Piper nigrum and Piper longum in viral hepatitis. The herb-containing composition of the present invention is therefore useful to prevent, treat or alleviate the symptoms of stress and/or infection in a subject.

Piper longum is a slender aromatic climber that is found all over India. The fruit of the herb has a pungent and sweet taste. Not only is P. longum useful as a spice, but it also has medicinal qualities as well. The biologically active constituents of P. longum include, e.g., piperine and piplartine. This herb has nerve depressant and antagonistic effects on electro-shock and chemo-shock seizures as well as other muscular spasms. It also has analgesic, tonic, stimulant, and carminative properties, and the oil of Piper longum has antibacterial properties. P. longum is most commonly used to treat respiratory infections such as cough, bronchitis, stomachache, diseases of the spleen, tumors, and asthma. When applied topically, it soothes and relieves muscular pains and inflammation. P. longum helps stimulate the appetite and it dispels gas from the intestines. The herb-containing composition of the present invention is therefore useful to prevent, treat or alleviate the symptoms of stress and/or infection in a subject.

P. nigrum (a.k.a., black pepper, pepper, white pepper, green peppercorns, Maricha, Gulmirch, Kurumulaku, Nellamulaku) is produced by a woody, broad-leaved evergreen vine that is cultivated today in many tropical lands, from India, Indonesia, and Malaysia to South America and the West Indies. The stout vine, which is allowed to climb poles or small trees in cultivation, bears many slender, densely packed flower spikes. The fruits that develop upon these spikes are generally harvested while still green; the signal is the reddening of the lowest fruits on a spike. The green fruits are dried until the flesh around the single hard seed is wrinkled and grayish black, then ground into black pepper or packaged and sold as whole peppercorns.

Black pepper's aromatic, slightly musty odor comes from the volatile oils found largely in the flesh and skin; its pungent bite comes from the alkaloids-piperine and piperidine-and resins found mostly in the seeds. The oils go into perfumes and flavorings. The biologically active constituents of Piper nigrum include, e.g., piperine, chavicine, piperidine, and piperettine. Black pepper is useful as an anthelmintic, antipyretic, antiperiodic, carminative, expectorant, and stimulant. It is useful in the treatment or alleviation of asthma, boils, cholera, colic, cough, diarrhea, fever, gas, gastric ailments, hemorrhoids, indigestion, chronic rheumatism, obesity, plegm, sinus congestion, skin diseases, sore throat, worms. The herb-containing composition of the present invention is therefore useful to prevent, treat or alleviate the symptoms of stress and/or infection in a subject.

Preparation and Standardization of the Herbal components of the Compositions of the Invention

A. A. paniculata Extract

In one embodiment, the A. paniculata extract of the herb-containing composition of the invention is derived from the whole plant of the A. paniculata herb and standardized for andrographolide content. Briefly, the A. paniculata herb was dried and then an aqueous extract was obtained using standard aqueous extraction technique known in the art. The extract was concentrated, the andrographolide content measured by standard chromatographic technique, and then the concentrate was standardized to andrographolide content by titration to required andrographolide content.

B. O. sanctum Exract and Powder

i. O. sanctum Exract

In one embodiment, the O. sanctum extract preparation of the herb-containing composition of the invention is derived from the leaves of the O. sanctum herb and standardized for ursolic acid content. Briefly, the leaves of the O. sanctum herb were collected, dried and then aqueous extracted using standard aqueous extraction techniques known in the art. The extract was concentrated, the ursolic content measured by standard chromatographic techniques, and then the concentrate was standardized to ursolic acid content by titration to required ursolic acid content.

ii. Holy Basil (O. sanctum) Powder

In one embodiment, the O. sanctum powder preparation of the herb-containing composition of the invention is derived from the leaves of the O. sanctum herb. Briefly, the leaves of the O. sanctum herb were collected, dried and then ground into a powder using techniques known in the art.

C. W. somnifera Extract

In one embodiment, the W. somnifera extract preparation of the herb-containing composition of the invention is derived from the root of the W. somnifera herb and standardized for withanolides content. Briefly, the root of the W. somnifera herb was collected, dried and then aqueous extracted using standard aqueous extraction techniques known in the art. The extract was concentrated, the withanolides content measured by standard chromatographic techniques, and then the concentrate was standardized to withanolide content by titration to required withanolide content.

D. Trikatu Extract

In one embodiment, the Trikatu extract preparation of the herb-containing composition of the invention is derived from the ‘three pungent spices’. (Dash V B, Junius A M M (1983). A Handbook of Ayurveda. New Delhi, India: Concept Publishing, pp. 195-196) and to enhance digestion and appetite (Simon D (1997). The Wisdom of Healing. New York, N.Y.: Harmony Books, p. 149). Specifically, the Trikatu extract is prepared as a 10:01 extract by standard techniques known in the art as an herbal mixture of equal parts of Long pepper fruit (Piper longum), Black pepper fruit (Piper nigrum), and Ginger root (Zingiber officinale).

Pharmaceutical Compositions and Formulations

The herb-containing compositions of the present invention can be used alone or further formulated with pharmaceutically acceptable compounds, vehicles, or adjuvants with a favorable delivery profile, i.e., suitable for delivery to a subject. Such compositions typically comprise the herb-containing composition of the invention and a pharmaceutically acceptable carrier. As used herein, “pharmaceutically acceptable carrier” is intended to include any and all solvents, dispersion media, coatings, antibacterial and antifungal compounds, isotonic and absorption delaying compounds, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include, e.g., oral; transdermal (i.e., topical), and transmucosal administration. The pH can be adjusted with acids or bases, such as hydrochloric acid or sodium hydroxide.

Oral compositions generally include an inert diluent or an edible carrier. They can be enclosed in gelatin capsules, caplets or compressed into tablets. For the purpose of oral therapeutic administration, the herb-containing composition of the invention can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring. The herb-containing compositions of the present invention can also be formulated as a topical cream for transdermal or transmucosal administration.

In one embodiment, the herb-containing compositions of the invention are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc.

The pharmaceutical compositions can be included in a container, pack, or dispenser together with instructions for administration.

The invention is further defined by reference to the following examples, which are not meant to limit the scope of the present invention. It will be apparent to those skilled in the art that many modifications, both to the materials and methods, may be practiced without departing from the purpose and interest of the invention.

### EXAMPLES

### Example 1

### Clinical Trial of Herb-Containing Tablet to Alleviate the Symptoms of Stress

General

Studies are conducted to investigate the effectiveness of a herb-containing composition in relieving the symptoms of stress. The test composition is a natural herb-containing preparation formulated as a tablet. The test composition contains standardized A. paniculata extract (150 mg/tablet; 30 mg equiv. andrographolides), standardized O. sanctum extract (175 mg/tablet; 3.5 mg equiv. ursolic acid), standardized W. somnifera extract (100 mg/tablet; 5 mg equiv. withanolides), Trikatu extract (25 mg/tablet) and holy basil leaf powder (50 mg/tablet).

The overall purpose of the proposed study is to determine whether a standardized herb-containing composition of the invention described above (RHI-174) improves psychosocial functioning, quality of life, and somatic health among subjects. A randomized double-blind, placebo-controlled parallel group clinical trial is performed to investigate the effect of RHI-174 in the treatment of stress. The study measures the effectiveness of RHI-174 in reducing the prevalence and intensity of symptoms and signs of stress as compared with a placebo. Specifically, the effect of RHI-174 on psychosocial functioning (perceived stress, coping patterns, social support, psychological distress), quality of life (including spiritual well-being), neuroendocrine mediation (cortisol, DHEA levels), and somatic health (number of colds, immune status) is assessed. A group of adult human test subjects of both sexes complete the randomized double-blind study. The test subjects are divided in two equal size groups, one of which received RHI-174 as 500 mg oral dose that is administered to the test subject at least once-daily after meals and the other group is administered a placebo during a period of up to 1 year. Evaluations for efficacy are performed by the test subjects at monthly intervals, i.e., 0, 1, 2, 5 months, etc., of the treatment. A decrease in the of any one stress symptom observed for the RHI-174 treatment group compared with placebo group or a decrease in the number of colds observed during the test period indicates that the RHI-174 composition is effective to reduce the prevalence and intensity of the stress symptoms leading to increased susceptibility to infection.

### Example 2

### Clinical Trial of Herb-Containing Tablet to Alleviate the Symptoms of Infection

General

Studies are conducted to investigate the effectiveness of a herb-containing composition in relieving the symptoms of infection. The test composition is a natural herb-containing preparation formulated as a tablet. The test composition contains standardized A. paniculata extract (150 mg/tablet; 30 mg equiv. andrographolides), standardized O. sanctum extract (175 mg/tablet; 3.5 mg equiv. ursolic acid), standardized W. somnifera extract (100 mg/tablet; 5 mg equiv. withanolides), standardized Trikatu extract (25 mg/tablet) and holy basil leaf powder (50 mg/tablet).

A randomized double-blind, placebo-controlled parallel group clinical trial is performed to investigate the effect of a standardized herb-containing composition of the invention described above (RHI-174) in the treatment of common cold infections. Trial design, patient scoring and analysis of results is similar to the clinical trial described by Caceres and coworkers. Caceres D. D. et al., Phytomedicine. 1999; 6:217-23. The objective of the study is to measure the effectiveness of RHI-174 in reducing the prevalence and intensity of symptoms and signs of common cold as compared with a placebo. A group of adult human test subjects of both sexes complete the randomized double-blind study. The patients are divided in two equal size groups, one of which receives RHI-174 as 500 mg oral dose three-times daily which they ingest after meals (total of 1500 mg oral dose per day) and the test subjects in the other group ingests a placebo during a period of 5 days. Evaluations for efficacy are performed by the test subjects at day 0, 2, and 4 of the treatment; each complete a self-evaluation (VAS) sheet with the following parameters: headache, tiredness, earache, sleeplessness, sore throat, nasal secretion, phlegm, frequency and intensity of cough. In order to quantify the magnitude of the reduction in the prevalence and intensity of the signs and symptoms of common cold, the risk (Odds Ratio=OR) is calculated using a logistic regression model. A decrease in the intensity of any one symptom observed for the RHI-174 treatment group compared with placebo group indicates that the RHI-174 composition is effective to reduce the prevalence and intensity of the symptoms in common cold infection.

### EQUIVALENTS

While the invention has been described in connection with the specific embodiments thereof, it will be understood that it is capable of further modification. Furthermore, this application is intended to cover any variations, uses, or adaptations of the invention, including such departures from the present disclosure as come within known or customary practice in the art to which the invention pertains, and as fall within the scope of the appended claims.

We claim:

1. An herb-containing composition, comprising herbal components which include a standardized Andrographis paniculata extract preparation with at least about 20% andrographolides based on total dry weight of the Andrographis paniculata extract preparation, a standardized Oculum sanctum extract preparation with at least about 2% ursolic acid based on total dry weight of the Oculum sanctum extract preparation, a standardized Withania somnifera extract preparation with at least about 5% withanolides based on total dry weight of the Withania somnifera extract preparation, a Trikatu extract, and a holy basil leaf powder, wherein the herb-containing composition is formulated as a dosage unit form as detailed in Table 1.

2. A method of preventing a symptom of stress in a subject, the method comprising administering to the subject in which prevention of the symptom of stress is desired the composition of claim 1 in an amount sufficient to prevent the symptom of stress.

3. A method of treating a symptom of stress in a subject, the method comprising administering to the subject in which treatment of the symptom of stress is desired the composition of claim 1 in an amount sufficient to treat one or more symptom of stress.

4. A method of preventing a symptom of infection in a subject, the method comprising administering to the subject in which prevention of the symptom of infection is desired the composition of claim 1 in an amount sufficient to prevent the symptom of infection.

5. A method of treating a symptom of infection in a subject, the method comprising administering to the subject in which treatment of the symptom of infection is desired the composition of claim 1 in an amount sufficient to treat one or more symptom of infection.
